(19) United States
(12) Patent Application Publication
(10) Pub. No.: US 2026/0083453 A1
(43) Pub. Date: Mar. 26, 2026

(54) SURGICAL INSTRUMENTS AND METHODS FOR USE

(71) Applicant: THE BOARD OF TRUSTEES OF THE LELAND STANFORD JUNIOR UNIVERSITY, Stanford, CA (US)

(72) Inventors: Y. Joseph Woo, Stanford, CA (US); Yuanjia Zhu, Stanford, CA (US); Matthew Park, Piedmont, CA (US)

(21) Appl. No.: 19/406,777

(22) Filed: Dec. 2, 2025

Related U.S. Application Data
(63) Continuation of application No. PCT/US24/32873, filed on Jun. 6, 2024.
(60) Provisional application No. 63/471,470, filed on Jun. 6, 2023.

(51) Int. Cl. A61B 17/04

(52) U.S. Cl. CPC A61B 17/0485

(57) ABSTRACT

Flexure based mechanisms for surgical instruments are provided, e.g., a knot pusher device including an elongate shaft including a proximal end and a distal end sized for introduction into a patient's body; and an end effector on the distal end comprising a knot pusher including a flexure mechanism for receiving a suture that prevents the suture from detaching once locked in and/or may provide haptic feedback to the user.

### RELATED APPLICATION DATA

The present application is a continuation of co-pending International Application No. PCT/US2024/032873, filed Jun. 6, 2024, which claims benefit of U.S. provisional application Ser. No. 63/471,470, filed Jun. 6, 2023, the entire disclosures of which are expressly incorporated by reference herein.

### STATEMENT REGARDING FEDERALLY SPONSORED RESEARCH AND DEVELOPMENT None.

### TECHNICAL FIELD

The present application relates to medical devices and, more particularly, to shafted surgical tools or instruments, e.g., knot pusher devices or other surgical instruments, and to methods for using such instruments.

### BACKGROUND

In the operating room, surgeons have a variety of actively controlled surgical devices. However, many of these devices are actively clamped and/or actuated using bulky mechanisms that can provide challenges for maneuvering the devices within small cavities.

For example, knot pushers may facilitate extracorporeal suturing across a broad range of suture sizes, and models are available in a variety of lengths and configurations, such as those shown in FIGS. 1A-1D. These devices are of utmost importance for cases that require precise knot tying operations in small, deep, minimally-invasive cavities. Many tools for pushing knots require a painstaking process of threading a suture through the eye of the tool or are designed in an open manner where the suture can easily fall off. Certain, more elaborate “trap model” devices make use of an actuation mechanism that allows the operator to more easily thread a suture through the tool while also securing the suture in place so as to prevent the suture from separating from the tool. However, these designs may be poorly suited to real minimally invasive conditions where they can easily get caught on other things, such as suture, tools, or even anatomy, in the operating field. This presents a significant risk, adding a vector for potential complication and/or increased time.

Accordingly, surgical tools that reduce such risks and/or facilitate extracorporeal suturing would be useful.

### SUMMARY

The present application is directed to medical devices and, more particularly, to shafted surgical tools or instruments, e.g., knot pusher devices or other surgical instruments, and to methods for using such instruments.

In one example, a knot pusher device is provided that allows for both efficient surgical tool performance, while also minimizing the risks associated with existing tools. The device may take advantage of the bending properties of elastic materials and/or precisely tuned geometries of flexure mechanisms to create one-way suture-locking designs. By utilizing flexure-based mechanisms and novel biocompatible materials, a new class of surgical devices may be provided that may serve a variety of advanced functions without the added costs associated with complex, multi-component actuated mechanisms. These flexure-based mechanisms may be widely applicable to a variety of different surgical tools beyond knot pushing and minimally invasive or laparoscopic operations.

For example, by integrating a flexure mechanism to non-clamping devices, otherwise bulky components of surgical instruments may be omitted, streamlining tooling, thereby allowing for much greater maneuverability of the tools within an operating cavity.

These flexure mechanisms may be tuned to provide optimal force feedback, and modular, variable catch/teeth designs may allow for a variety of different applications. In one particular example, the devices may facilitate knot tying descension into an operating cavity for a variety of surgical procedures, including, but not limited to, cardiac, thoracic, and otolaryngological operations (i.e., knot pushing).

In accordance with one example, a surgical instrument is provided that includes an elongated shaft including a proximal end and a distal end sized for introduction into a patient's body; and an end effector on the distal end comprising a knot pusher including a flexure mechanism for receiving a suture that prevents the suture from detaching once locked in. Optionally, the knot pusher may also provide haptic feedback to the user upon locking the suture to the end effector. For example, the end effector may include a hook and tongue defining an eyelet and a passage for introducing a suture into the eyelet, e.g., wherein the passage of the end effector has a cross-section smaller than a diameter of the eyelet, and wherein one or both of the hook and the tongue have sufficient flexibility to increase the cross-section of the passage to accommodate inserting a suture through the inlet and passage into the eyelet.

In accordance with another example, a surgical instrument is provided that includes an elongated shaft including a proximal end and a distal end sized for introduction into a patient's body; and an end effector on the distal end comprising a knot pusher including a hook and tongue defining an eyelet and one or more passages between the hook and the tongue for introducing a suture into the eyelet, one or both of the hook and base comprising a flexure mechanism for receiving a suture in the eyelet that prevents the suture from detaching once locked in the eyelet.

In accordance with another example, a surgical instrument is provided that includes an elongated shaft including a proximal end and a distal end sized for introduction into a patient's body; and an end effector on the distal end comprising a hook and tongue at least partially surrounding an eyelet and one or more passages between the hook and the tongue for introducing a suture into the eyelet, one or both of the hook and base comprising flexible material configured to resiliently deflect to expand the one or more passages from a closed position in which a cross-section of the one or more passages is smaller than a cross-section of the eyelet to an open position in which the cross-section of the one or more passages is increased to allow a suture sized to be received through the eyelet to pass through the one or more passages.

In accordance with yet another example, a surgical instrument is provided that includes an elongated shaft including a proximal end and a distal end sized for introduction into a patient's body; and a plurality of end effectors, each end effector configured to be coupled to the distal end of the shaft and comprising a knot pusher including a flexure mechanism defining an eyelet for receiving a suture that secures the suture to the end effector once received in the eyelet. Optionally, one or more of the end effectors may include eyelets having different diameters to accommodate different size sutures and/or may include one or more bends such that the knot pushers extend along an axis that intersects a longitudinal axis of the shaft.

In accordance with still another example, a system or kit is provided that includes a plurality of surgical instruments, each instrument comprising an elongated shaft including a proximal end and a distal end sized for introduction into a patient's body; and an end effector on the distal end comprising a knot pusher including a flexure mechanism defining an eyelet for receiving a suture that secures the suture to the end effector once received in the eyelet. Optionally, one or more of the surgical instruments may include shafts having different lengths between the proximal end and distal end, may include eyelets having different diameters to accommodate different size sutures, and/or may include end effectors including one or more bends such that the knot pushers extend along an axis that intersects a longitudinal axis of the shafts.

In accordance with another example, a method is provided for advancing a knot over a suture line to a surgical site within a patient's body that includes providing an end effector comprising a hook and a tongue defining an inlet communicating with an eyelet; forming a knot on the suture line; inserting one end of the suture line through the inlet into the eyelet to lock the suture to the end effector; and advancing the end effector to push the knot to the surgical site.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A-1D show examples of ends of conventional knot pusher instruments.

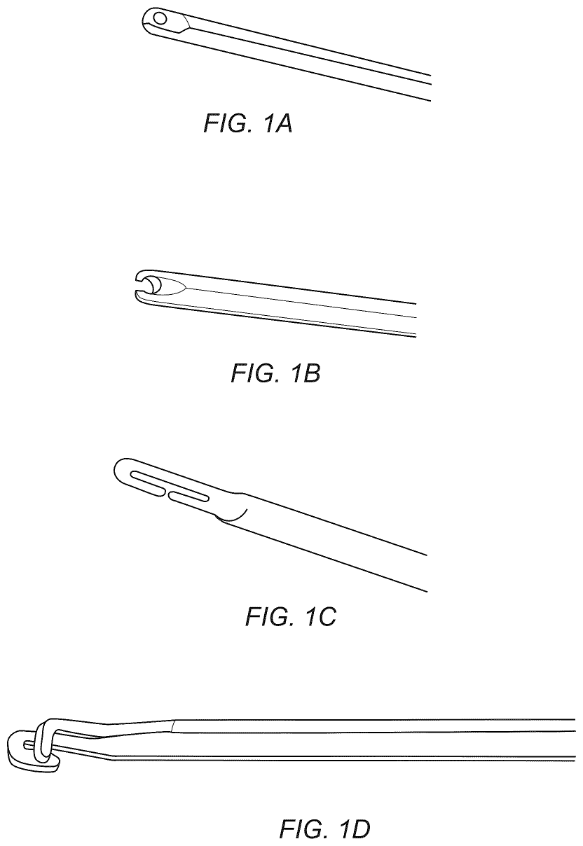

FIGS. 2A and 2B are side and perspective views, respectively, of an example of a surgical instrument including a handle portion and an end effector connected to the handle portion.

FIGS. 3A and 3B are perspective and side views, respectively, of the handle portion of the instrument of FIGS. 2A and 2B.

FIGS. 4A-4C are details of the end effector on the handle portion of the instrument of FIGS. 2A and 2B.

FIGS. 5A-5C are various views of an exemplary knot pusher end effector that may be coupled to the handle portion shown in FIGS. 3A and 3B.

FIGS. 6A and 6B are cross-sectional views of the end effector of FIGS. 5A-5C taken along a centerline plane of the end effector.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about. ” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

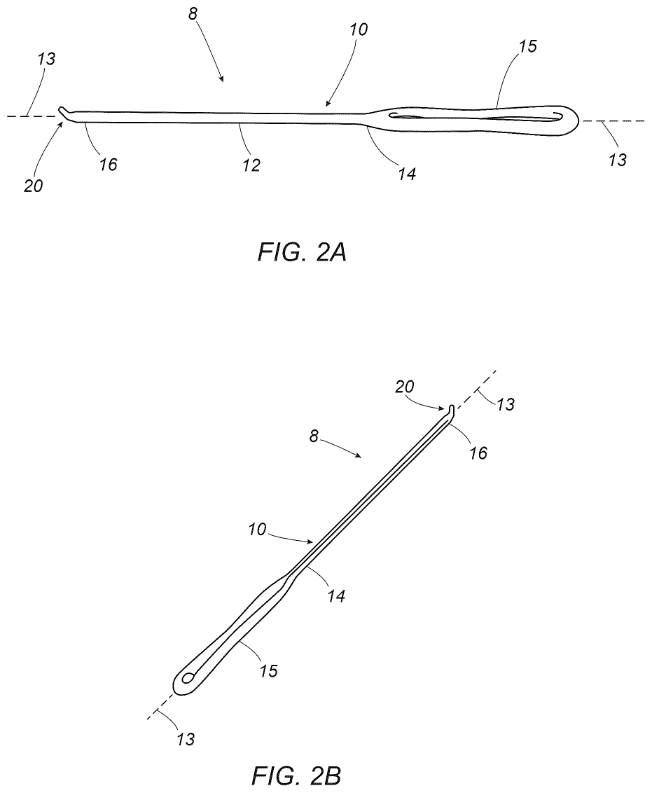

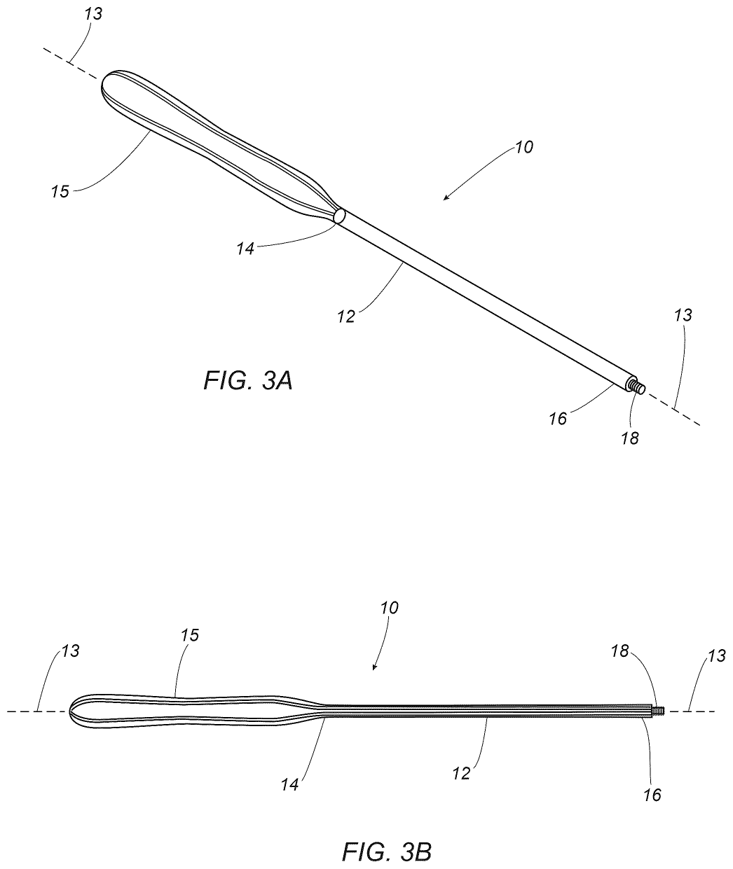

Turning to the drawings, FIGS. 2A and 2B show an example of a surgical tool, namely a knot pusher device or instrument 8 that includes a handle portion 10 and an end effector 20, e.g., removably connected to the handle portion 10. As best seen in FIGS. 3A and 3B, the handle portion 10 includes an elongated shaft 12 including a proximal end 14 with a grip 15 and a distal end 16 sized for introduction into a patient's body, e.g., through a port or other access (not shown). The shaft 12 may be substantially rigid between the proximal and distal ends 14, 16 or may be malleable along at least a portion thereof, e.g., at or adjacent to the distal end 16. For example, the handle portion 10 may be formed from one or more biocompatible materials, e.g., metal, such as stainless steel, plastic, or composite materials, e.g., by one or more of machining, casting, molding, laser cutting, and the like.

Optionally, a plurality of handle portions may be provided in a kit including different configurations and/or geometries, e.g., different shaft lengths and/or shapes (not shown), if desired. For example, as shown in FIGS. 3A and 3B, the shaft 12 may be substantially straight between the proximal and distal ends 14, 16, e.g., aligned along a longitudinal axis 13, although alternatively, if desired, the shaft 12 may include one or more bends or curves (not shown), e.g., adjacent the distal end 16 to orient the end effector 20 in a desired manner relative to the grip 15. Also as shown, the grip 15 is a substantially straight handle, e.g., aligned with the longitudinal axis 13, although alternatively, the grip may be oriented transversely relative to the axis and/or may have other shapes that facilitate holding and/or manipulating the device 8 during a particular procedure. Alternatively, a hub or other connector may be provided in place of the grip 15 on the proximal end 14, e.g., such that the shaft 12 may be connected to an arm or other actuator of a robotic or other remotely operable surgical system (not shown), if desired, as described elsewhere herein.

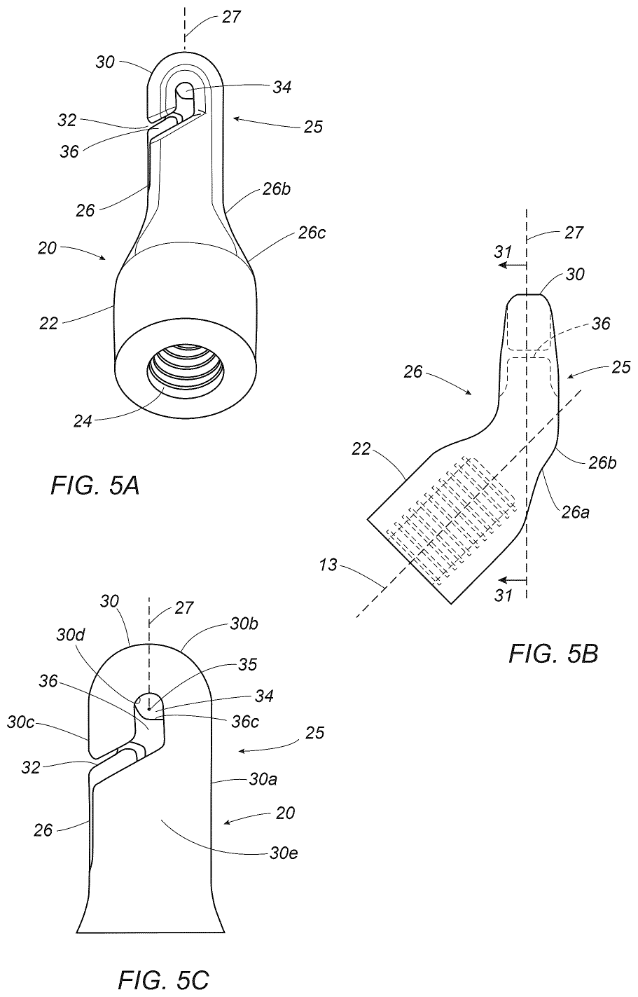

As shown in FIGS. 3A and 3B, a connector 18 may be provided on the distal end 16, which may be configured to removably secure an end effector 20 to the distal end 16. For example, as shown, the distal end 16 includes a threaded hub 18 configured to be received in a corresponding threaded recess 24 of an end effector 20, such as the end effector 20 shown in FIGS. 5A-5C. Alternatively, other connectors and/or ends may be provided on the distal end 16, e.g., including one or more detents, recesses, annular grooves, snap fittings, and the like, that are configured to couple to a corresponding end effector. Optionally, a plurality of end effectors may be provided in a kit having different configurations (not shown), and one of the end effectors may be selected and coupled to the distal end 16 for use during a particular procedure.

Alternatively, one or more instruments may be provided that include an end effector permanently coupled to a handle portion (not shown). For example, a plurality of instruments may be provided in a kit that include different shaft configurations, e.g., straight, bent, curved, and the like, with the same or different size end effectors, in a variety of combinations to provide a kit for a surgical procedure.

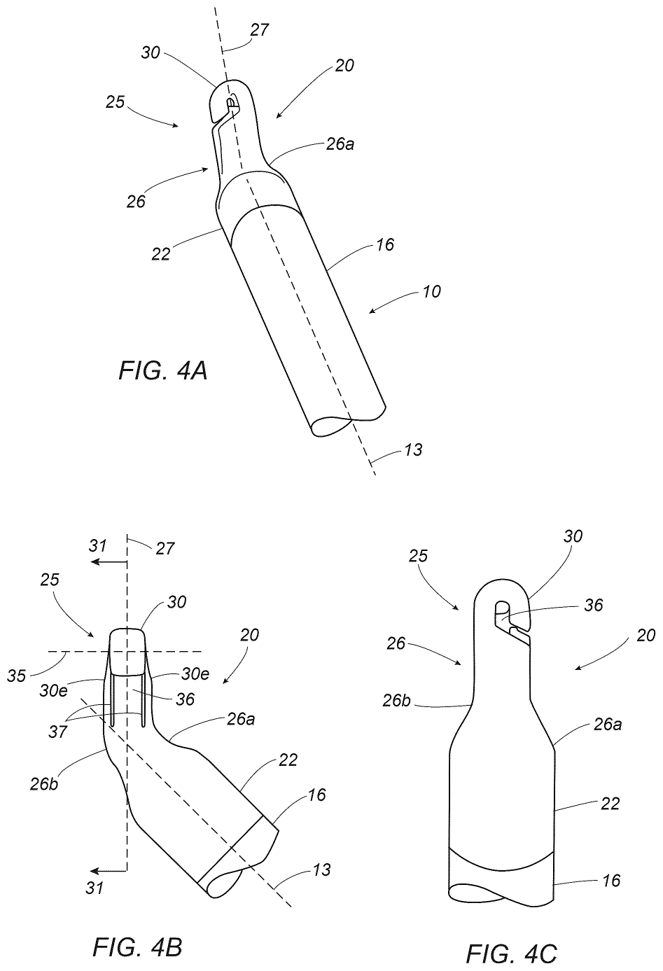

FIGS. 4A-4C show an end effector 20 connected to the distal end 16 of a handle portion 10, which may be similar to the handle portion 10 shown in FIGS. 3A and 3B or as otherwise described elsewhere herein. In the example shown, the end effector 20 includes a hub or coupling portion 22 that may be removably coupled to the distal end 16 of the handle portion 10. For example, as shown in FIGS. 5A and 5B, the hub 22 may include a threaded recess 24 corresponding to the threaded hub 18 on the handle portion 10 shown in FIGS. 3A and 3B. Thus, the end effector 20 may be connected to the handle portion 10 simply by threading the hub 18 into the recess 24 to securely but removably couple the end effector 20 to the handle portion 10. Alternatively, the hub 22 may include one or more other connectors (not shown) that may be removably or permanently coupled to corresponding connector(s) on the distal end 16 of the handle portion 10, as described elsewhere wherein.

A flexure or tool member 25 may extend from the hub 22 that is configured to receive or otherwise selectively secure an elongate member, such as a suture or other surgical element (not shown), e.g., to facilitate manipulating the surgical element during a procedure. For example, as shown, the flexure member 25 includes a base 26 extending from the hub 22 that includes a hook 30 and tongue 36 that together define one or more passages 33 communicating with an eyelet 34 into which a suture or other surgical element may be removably inserted during a procedure.

The base 26 may extend from the hub 22 at an angle relative to the longitudinal axis 13, e.g., along a hub axis 27. In the example shown, the hub 26 may include a tapered region 26a extending from the hub 22 and a bend 26b such that the hub axis 27 and a centerline plane 31 of the hook 30, e.g., as shown in FIGS. 4B and 5B, intersects the longitudinal axis 18 at an acute angle, e.g., between about. Alternatively, the base 26 may be aligned along the longitudinal axis 13 and/or may have other curved or other nonlinear shapes, as desired.

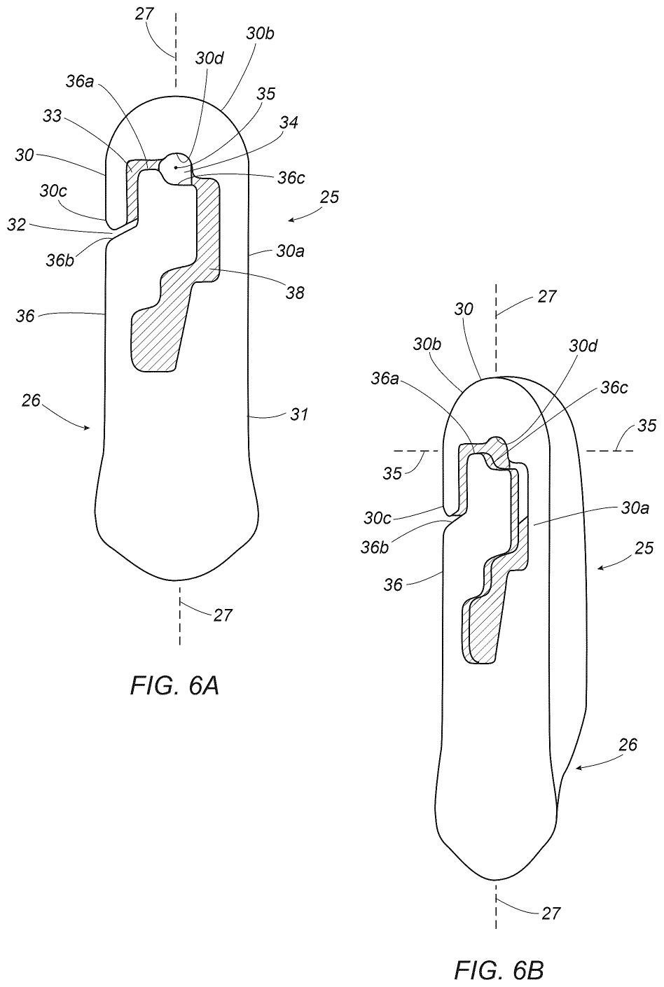

With further reference to FIGS. 5A-6B, the flexure member 25 may provide a knot pusher end effector with the hook 30 and tongue 36 together defining a set of passages 33 for receiving a suture (not shown) into an eyelet 34. As best seen in FIGS. 6A and 6B, the tongue 36 may extend from the base 26 generally along the hub axis 27 within the centerline plane 31 (e.g., defined by the surface shown in FIG. 6A) terminating in a distal tip 36a. The hook 30 may at least partially surround the tongue 36, e.g., including a first portion 30a extending from the base 26 adjacent the tongue 26 and a curved hood portion 30b extending from the first portion 30a and at least partially surrounding the distal tip 36a of the tongue 36. For example, the hood portion 30b may curve from the first portion 30a around the tip 36a of the tongue and terminate at a hook tip 30c adjacent a shoulder 36c of the tongue 36, thereby defining an inlet 32 between the hook tip 30c and shoulder 36c that communicates with the passage(s) 33 to the eyelet 34. The hood portion 30b may have a rounded distal surface, e.g., to provide a substantially atraumatic distal tip for the end effector 20, which may facilitate advancing the end effector 20 to a surgical site and/or otherwise manipulating the end effector 20, while avoiding damaging contacted tissue.

Optionally, the hook 30 may at least partially surround the tongue 36 between the hub 26 and the distal tip 36a. For example, as best seen in FIGS. 4A-4C and 5C, the hook 30 may include side portions 30e on opposite sides of the tongue 36, e.g., extending from the first portion 30a of the hook 30, thereby defining longitudinal slots 37 on either side of the tongue 36 (shown in FIG. 4B). Thus, the first portion 30a and side portions 30e may define three sides of the flexure member 25 and the tongue 36 may define a fourth side between the slots 37.

The eyelet 34 may be at least partially defined by surfaces of the hook 30 and tongue 36 such that the eyelet 34 appears as an enclosed opening aligned along an eyelet axis 34, e.g., substantially perpendicular to the centerline plane 31 of the hook 30 and/or the hub axis 27. However, the surfaces do not entirely enclose the eyelet 34 but provide spaces such that the passage(s) 33 communicate with the eyelet 34.

For example, as best seen in FIGS. 5C and 6A, the eyelet 34 may be defined by a pair of concave inner surfaces 30d on the hood portion 30b of the hook 30 and a concave tip surface 36c on the tip 36a of the tongue 36. The concave tip surface 36c may be positioned opposite and between the inner surfaces 30d on the hook 30 to define a substantially circular opening aligned along an eyelet axis 35 orthogonal to the hub axis 27.

When viewed along the eyelet axis 35, e.g., as shown in FIGS. 5C and 6A, the eyelet 34 may define a substantially enclosed opening having a diameter or other cross-section sized to provide sufficient clearance such that, once a suture (or other elongate surgical element, not shown) is received within the eyelet 34, the end effector 20 may be advanced and/or retracted over the suture with minimal resistance. For example, the eyelet 34 may have a cross-section to accommodate slidably receiving a variety of size sutures, e.g., between about 0.35-0.40 mm, 0.30-0.34 mm, 0.20-0.25 mm, 0.15-0.20 mm, 0.10-0.15 mm, 0.07-0.10 mm, 0.05-0.07 mm, 0.04-0.05 mm, or 0.03-0.04 mm. Optionally, a plurality of end effectors 20 may be provided including different size eyelets and/or different flexibility characteristics, such that an operator may select an end effector having a desired configuration for a particular suture (or other surgical element) being manipulated during a procedure.

As best seen in FIGS. 6A and 6B, the hood portion 30b of the hook 30 and the tip 36a of the tongue 36 may be spaced apart to provide passages 33 extending from the inlet 32 to the eyelet 34 such that a suture (not shown) may be inserted into the inlet 32, e.g., substantially parallel with the eyelet axis 34, and through the passages 33 until the suture is received through the eyelet 34 for introduction into and/or manipulation within a patient's body, as explained further elsewhere herein.

Optionally, the passages 33 may be sized for a specific diameter of suture, e.g., to allow the suture to be introduced easily into the eyelet 34 while preventing the suture from detaching once locked in, e.g., to facilitate introducing the suture to a surgical site. For example, as shown, the passage 33 may have a cross-section smaller than the diameter of the eyelet 34, and one or both of the tongue 36 and the hook 30 may have sufficient flexibility to allow the cross-section of the passages 33 to be increased, e.g., to accommodate inserting a suture into the inlet 32, through the passages 33, and into the eyelet 34.

In one example, the hood portion 30b may be substantially rigid and the tongue 36 may be resiliently deflectable. For example, one or more inner passages 36d may be provided within the end effector 20, e.g., between the tongue 36 and the first portion 30a of the hook 30, such that the tongue 36 may provide a hinge or cantilevered member, allowing the tip 36a of the tongue 36 to deflect inwardly into the inner passage(s) 36d to increase the cross-section of the inlet 32 and/or passages 33. The material of the tongue 36 may provide sufficient flexibility to accommodate deflection of the tongue 36, e.g., during insertion of the suture, yet be biased to resiliently deflect the tongue 36 back outwardly to decrease the cross-section of the passages 33, thereby locking the suture within the eyelet 34. In this option, the user may be able to press the tongue 36 inwardly, e.g., by pressing against the shoulder 36c (or outer surface of the tongue 36 between the slots 37) to move the tongue 36 from a closed or outward position to an open or inward position to facilitate inserting the suture, with the tongue 36 automatically returning to the closed position when released.

Alternatively, the tongue 36 may be substantially rigid, and the hood portion 30b of the hook 30 may sufficiently flexible to allow the hook tip 30c to move away from the shoulder 36b of the tongue 36 to open the inlet 32 sufficiently to allow a suture to enter the inlet 32. Similarly, the hood portion 30b may be moved away from the tip 36a of the tongue 36 to allow the suture to slide through the passages 33 until positioned in the eyelet 34. Thus, the hook 30 may be biased to a closed position yet resiliently moved to an opened position to accommodate inserting the suture when the operator applies sufficient force to the suture to open the inlet 32 and passages 33. In a further alternative, both the hook 30 and the tongue 36 may be flexible to move between closed positions where the cross-sections of the inlet 32 and passages 33 are smaller than the eyelet 34 (and smaller than the diameter of the suture) and open positions to allow insertion of the suture into the eyelet 34. Thus, with the hook 30 and/or tongue 36 biased to the closed positions, the suture may be pressed into the inlet 32, through the passages 33, and locked into the eyelet 34 when the material resiliently returns to the closed position, preventing the suture from sliding back through the passages 33 and out the inlet 32. At any time, the operator may push the suture back through the passages 33 and out the inlet 32 by overcoming the bias of the flexible material of the hook 30 and/or tongue 36.

In one example, the end effector 20 may be manufactured using one or more biocompatible materials, e.g., formed by one or more of 3D-printing, molding, casting, machining, laser cutting, and the like to provide the desired geometry of the components of the end effector 20. The material(s) may be selectively tuned to provide the right amount of locking resistance to the hook 30 and/or tongue 36 for a specific suture diameter, preventing the suture from detaching once locked in, while optionally providing haptic feedback to the operator indicating that the suture has been locked within the eyelet 34. For example, the end effector 20 may be formed from a rigid, semi-rigid, or flexible material, e.g., biased to a relaxed shape or configuration, but having sufficient flexibility to facilitate insertion, manipulation, and release of a suture during a surgical procedure. Since the device utilizes a flexure mechanism, in one example the end effector may be manufactured as a single body that may be passively actuated, which may convey advantages including a reduction of components and simplicity in operation.

During use during a surgical procedure, a suture may be secured to a surgical site, e.g., such that ends of the suture extend from the surgical site to a location outside the patient's body. For example, a needle or other suturing device (not shown) may be introduced through a port or other access to the surgical site to deliver a suture through tissue (and/or through a prosthesis, implant, or other device positioned adjacent the tissue), e.g., using conventional methods.

To advance a knot over the suture, a knot may be formed on the suture and one end of the suture may be inserted through the inlet 32 and passages 33 into the eyelet 34. For example, as explained above, the tongue 36 and/or hook 30 may be sufficiently flexible to open the passages 33 until the suture is received within the eyelet 34, whereupon the passages 33 may close to lock the suture within the eyelet 34.

The end effector 20 may then be advanced over the suture to advance the knot to the surgical site. For example, the surgeon holding the grip 15 of the instrument 8 may manually advance the shaft 12 to direct the end effector 20 and knot to the surgical site. Given that the knot is larger than the eyelet 34, the knot may slide down the suture as the end effector 20 is advanced. Once the knot is secured in place, the end effector 20 may be withdrawn back over the suture, e.g., by manually withdrawing the instrument 8.

The suture may then be withdrawn from the eyelet 34, e.g., by again deflecting the tongue 36 and/or hook 30 or applying sufficient force to direct the suture through the passages 33 out the inlet 32. If desired, another knot may be formed, the suture inserted into the eyelet 34, and the knot advanced to the surgical site by again advancing the instrument 8. This process may be repeated as many times as desired to create one or more additional knots on a single suture or multiple sutures.

In the example shown in FIGS. 2A and 2B, the end effector 20 and instrument 8 may have particular utility for minimally invasive, neochordal mitral valve repair operations, where it has been shown that the precise lengths of neochordae (which are usually just Gore-Tex suture) have significant, profound impacts on the quality of the repair and outcomes. These neochordae are usually tightened using knot pushing surgical tools, and recent studies have shown that millimeter level mis-estimations of neochord lengths can increase the forces on the valve, which, when compounded by the cyclic loading of the heart, can be detrimental to the long-term performance of the valve.

Alternatively, the end effector device may be modified for transcatheter use, e.g., by coupling the end effector 20 to a distal end of a flexible shaft that may be introduced via a catheter, sheath, or other tubular member (not shown). In this option, the operator may form a knot outside the patient's body, insert the suture into the eyelet 34, and advance the shaft to advance the knot to the target location. Thus, a suture may be positioned from a target surgical site through one or more blood vessels to an access site, and the end effector 20 may be advanced over the suture, e.g., to push a knot through the vessels to the surgical site. In another alternative, the end effector 20 may be provided on a robotic arm or other remotely operable surgical apparatus, which may be used to advance one or more knots along a suture received with the eyelet 34.

Moreover, the flexure-based mechanism of the devices herein may be broadly applicable to a whole class of surgical devices including but not limited to minimally invasive, laparoscopic, and/or transcatheter devices, beyond knot pushing. Potential further uses for this specific device include robotic surgeries where the device may be used to tie knots through a robotic port inside of the body cavity, and laparoscopic procedures where similarly suture tying is needed inside of the body. The device may allow a knot to be pushed down through the working ports without the need to bring the entire suture including the needle to the inside of a body cavity to type with instruments, which can be challenging, slow, less secure, and with risk of retaining foreign materials such as remaining sutures or even needles.

Further, although the devices are described with particular focus on advancing knots over sutures, it will be appreciated that the flexure mechanisms may be used to manipulate a variety of elongate surgical elements, which may be inserted into and locked into the eyelet to allow manipulation, and then removed from the eyelet to release the surgical element.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A surgical instrument, comprising:
an elongated shaft including a proximal end and a distal end sized for introduction into a patient's body; and
an end effector on the distal end comprising a knot pusher including a flexure mechanism for receiving a suture that prevents the suture from detaching once locked in the flexure mechanism.

2. The instrument of claim 1, wherein the end effector comprises a hook and a tongue defining an eyelet and one or more passages for introducing a suture into the eyelet between the hook and the tongue.

3. The instrument of claim 2, wherein one or more passages of the end effector have a cross-section smaller than a diameter or other cross-section of the eyelet, and wherein one or both of the hook and the tongue have sufficient flexibility to increase the cross-section of the one or more passages to accommodate inserting a suture through the inlet and one or more passages into the eyelet.

4. The instrument of claim 3, wherein the tongue comprises a hinge configured to allow the tongue to deflect inwardly or outwardly to increase the cross-section of the one or more passages, the hinge biased to return outwardly or inwardly to decrease the cross-section of the one or more passages, thereby locking the suture once received in the eyelet.

5. The instrument of claim 4, wherein the hook is substantially rigid.

6. The instrument of claim 4, wherein the hook comprises flexible material to accommodate inserting a suture through the inlet and one or more passages into the eyelet.

7. The instrument of claim 2, wherein the hook comprises a hood portion that at least partially surround a distal tip of the tongue to define the one or more passages.

8. The instrument of claim 7, wherein the hood portion comprises a concave inner surface and the distal tip of the tongue comprises a concave surface that together at least partially define a perimeter of the eyelet.

9. The instrument of claim 1, wherein the end effector is removably coupled to the distal end of the shaft.

10. The instrument of claim 1, wherein the end effector is permanently coupled to the distal end of the shaft.

11. The instrument of claim 1, wherein the shaft is substantially rigid.

12. The instrument of claim 1, wherein at least the distal end of the shaft is malleable or flexible.

13. (canceled)

14. The instrument of claim 1, further comprising a grip on the proximal end of the shaft.

15. The instrument of claim 1, wherein the proximal end of the shaft is connectable to a robotic surgical system.

16. The instrument of claim 15, wherein the shaft is flexible between the proximal and distal ends for transcatheter introduction into a patient's body.

17-35. (canceled)

36. A kit comprising a plurality of surgical instruments, each instrument comprising:
an elongated shaft including a proximal end and a distal end sized for introduction into a patient's body; and
an end effector on the distal end comprising a knot pusher including a flexure mechanism defining an eyelet for receiving a suture that secures the suture to the end effector once received in the eyelet.

37. (canceled)

38. The kit of claim 36, wherein one or more of the surgical instruments comprise eyelets having different diameters to accommodate different size sutures.

39. (canceled)

40. The kit of claim 36, wherein the end effector on each instrument comprises a hook and tongue at least partially surrounding the eyelet and one or more passages between the hook and the tongue for introducing a suture into the eyelet, one or both of the hook and base comprising flexible material configured to resiliently deflect to expand the one or more passages from a closed position in which a cross-section of the one or more passages is smaller than a cross-section of the eyelet to an open position in which the cross-section of the one or more passages is increased to allow a suture sized to be received through the eyelet to pass through the one or more passages.

41-60. (canceled)

61. A method for advancing a knot over a suture line to a surgical site within a patient's body, comprising:
providing an end effector comprising a tongue and a hook defining an inlet communicating with an eyelet;
forming a knot on the suture line;
inserting one end of the suture line through the inlet into the eyelet to lock the suture to the end effector; and
advancing the end effector to push the knot to the surgical site.

62-64. (canceled)
